(19) United States
(12) Patent Application Publication
(10) Pub. No.: US 2023/0355349 A1
(43) Pub. Date: Nov. 9, 2023

(54) MEDICAL DEVICE

(71) Applicant: TERUMO KABUSHIKI KAISHA, Tokyo (JP)

(72) Inventors: Takito INUKAI, Isehara Kanagawa (JP); Yuji ONIMURA, Fujinomiya Shizuoka (JP); Hiroshi SATO, Fuji Shizuoka (JP); Takayuki MORI, Chigasaki Kanagawa (JP)

(21) Appl. No.: 18/352,277

(22) Filed: Jul. 14, 2023

Related U.S. Application Data
(63) Continuation of application No. PCT/JP2021/045046, filed on Dec. 8, 2021.

(30) Foreign Application Priority Data
Jan. 18, 2021 (JP) 2021-005613

(51) Int. Cl. A61B 5/107; A61B 90/00

(52) U.S. Cl. CPC A61B 90/39; A61B 5/1072; A61B 2090/3937

(57) ABSTRACT

A medical device for assisting in a puncture operation on a human body, includes an imaging unit configured to acquire a cross-sectional image of a human body, a laser unit configured to emit laser light and project a marker onto a skin surface of the human body, a controller configured to: determine a first distance from the imaging unit to a blood vessel using the cross-sectional image acquired by the imaging unit, determine a position of a puncture point on the skin surface with respect to the imaging unit based on the determined first distance, and control the laser unit to project onto the skin surface a marker indicating the puncture point at the determined position thereof.

### CROSS-REFERENCE TO RELATED APPLICATION

This application is a continuation of International Pat. Application No. PCT/JP2021/045046 filed Dec. 8, 2021, which is based upon and claims the benefit of priority from Japanese Pat. Application No. 2021-005613, filed on Jan. 18, 2021, the entire contents of which are incorporated herein by reference.

### FIELD

The present disclosure relates generally to a medical device and a method for assisting in a puncture operation on a human body.

### BACKGROUND

Vascular puncture of puncturing a human body with an injection needle is performed in order to secure an access site for drug administration and endovascular treatment. In the vascular puncture, it is difficult for an operator to visually observe a blood vessel from a skin surface, and thus, a position of the blood vessel is guessed using standard knowledge of blood vessel locations and skill such as tactile perception of blood vessel pulsation. However, failure in the vascular puncture often occurs, which causes physical and mental distress to a patient.

In recent years, techniques for visualizing a blood vessel position, such as a near-infrared image, an ultrasound echo, and photoacoustic imaging, are sometimes used in order to specify a puncture position. For example, there is a known technique for displaying a cross-sectional image of an arm on a monitor using an ultrasound echographic apparatus.

Although the above-described technique for visualizing a blood vessel make it possible to specify the blood vessel location at the time of puncture, since a positional relationship between a visualized image and a skin surface is not always clear, certain skill is required to grasp a puncture position. In particular, in a puncture operation using the ultrasound echographic apparatus, the cross-sectional image acquired by the ultrasound echographic apparatus is displayed on the monitor, and it is necessary for an operator to guess a puncture location on the arm of a patient while viewing the cross-sectional image. In addition, a near-infrared image enables projection of a blood vessel image on the skin surface, but near-infrared rays are reflected and attenuated in the body, and thus, the accuracy of the position is low, and there is a high possibility that deviation from an actual blood vessel position occurs.

### SUMMARY OF THE INVENTION

Embodiments of this disclosure provide a medical device capable of indicating a position of a puncture point on a skin surface with high accuracy.

A medical device for assisting in a puncture operation on a human body, comprises an imaging unit configured to acquire a cross-sectional image of a human body, a laser unit configured to emit laser light and project a marker onto a skin surface of the human body, and a controller configured to: determine a first distance from the imaging unit to a blood vessel using the cross-sectional image acquired by the imaging unit, determine a position of a puncture point on the skin surface with respect to the imaging unit based on the determined first distance, and control the laser unit to project onto the skin surface a marker indicating the puncture point at the determined position thereof.

Since the medical device configured as described above specifies the position of the blood vessel using the cross-sectional image acquired by the imaging unit and projects the marker indicating the puncture point on the skin surface, an operator does not need to move the line of sight to a monitor or the like, and can concentrate on the puncture operation, so that the puncture can be reliably performed regardless of the skill of the operator.

### BRIEF DESCRIPTION OF THE DRAWINGS

FIG. 1 is a front view of a blood vessel position indicating device in an embodiment.

FIG. 2 is a side view of the blood vessel position indicating device.

FIG. 3 is a view illustrating a lower surface of the blood vessel position indicating device and illustrating a positional relationship with an arm from which a cross-sectional image is acquired.

FIG. 4 is a front view of the blood vessel position indicating device performing a projection operation on a skin surface of the arm.

FIGS. 5A to 5C depict marks projected on the skin surface and indicating different puncture depths and different blood vessel diameters.

FIGS. 6A to 6C are views illustrating a plurality of patterns of a direction indicator.

FIGS. 7A to 7D depict different marks projected on the skin surface.

FIG. 8 is a hardware block diagram of the blood vessel position indicating device.

FIG. 9 is a view illustrating an example of an image acquired by an imaging unit.

FIG. 10 is a view illustrating a positional relationship among a position of the center of gravity of a blood vessel, an imaging position, and a puncture point in a state in which a probe body is parallel to a skin surface.

FIG. 11 is a view illustrating a positional relationship among an arm holding portion, the arm, and the blood vessel position indicating device.

FIG. 12 is a view illustrating a positional relationship among the position of the center of gravity of the blood vessel, the imaging position, and the puncture point in a state in which the probe body is inclined with respect to the skin surface.

FIG. 13 is a view illustrating a positional relationship among the position of the center of gravity of the blood vessel, the imaging position, and the puncture point in the state in which the probe body is parallel to the skin surface.

FIG. 14 is a view illustrating a positional relationship among the position of the center of gravity of the blood vessel, the imaging position, and the puncture point in a state in which the probe body is inclined with respect to the skin surface.

### DETAILED DESCRIPTION

Hereinafter, embodiments of the present invention will be described with reference to the drawings. Note that dimensional ratios of the drawings are exaggerated for the convenience of description and may differ from actual ratios in some cases.

A blood vessel position indicating device 10 according to an embodiment is a medical device used by an operator who performs a puncture operation on an arm of a human body. The blood vessel position indicating device 10 acquires a cross-sectional image of the arm to detect a blood vessel position, and projects a puncture position calculated based on the blood vessel position onto a skin surface.

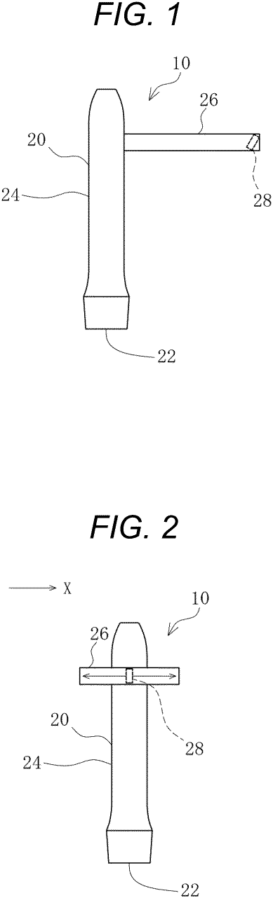

As illustrated in FIGS. 1 and 2, the blood vessel position indicating device 10 includes a probe body 20 having an imaging unit 22 that comes into contact with the skin surface to acquire the cross-sectional image of the human body, and an extension portion 26 protruding in one direction from an upper portion of the probe body 20. A laser unit 28 includes one or more laser diodes that emits laser light obliquely downward and is provided at a distal end of the extension portion 26. The distal end of the extension portion 26 is formed to be wide along X direction as shown in FIG. 2, and the laser unit 28 can be manually moved by the operator along the X direction in the extension portion 26.

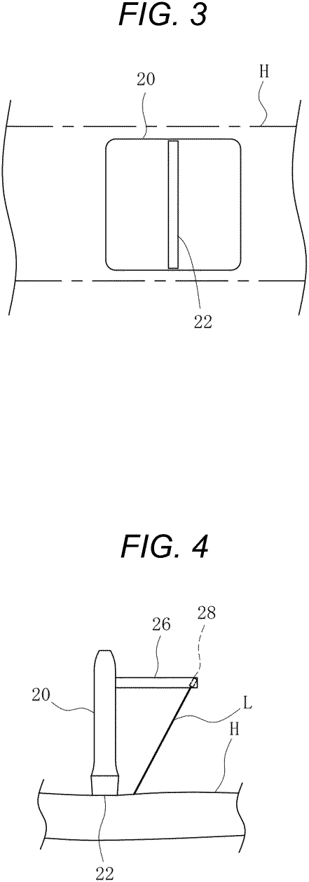

The probe body 20 has the imaging unit 22 at a lower end, and has a vertically long handle portion 24, gripped by an operator, in the upper portion. The handle portion 24 may be attached to a robot for performing a puncture operation. When the handle portion 24 is attached to the robot, the handle portion 24 may have a shape other than the vertically long shape. As illustrated in FIG. 3, the imaging unit 22 is provided so as to extend over substantially the entire width at the central portion of a lower surface of the probe body 20. The imaging unit 22 is an echographic apparatus that includes a transducer that generates an ultrasound wave and obtains the cross-sectional image of the inside of the human body by detecting a reflected wave of the ultrasound wave. The cross-sectional image orthogonal to the axial direction of a blood vessel is acquired, and thus, the imaging unit 22 is arranged such that the length direction thereof is orthogonal to the length direction of an arm H.

In a state where the imaging unit 22 is in contact with a skin surface of the arm H as illustrated in FIG. 4, the blood vessel position indicating device 10 causes the laser unit 28 to emit laser light L toward the skin surface to project a marker indicating a position of a puncture point. In this example, an irradiation angle of the laser light with respect to a perpendicular line of the skin surface is constant at 30°.

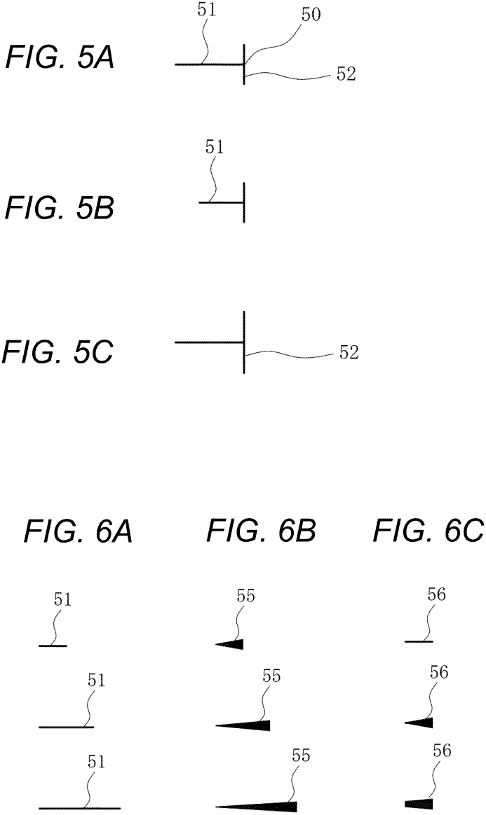

The marker projected on the skin surface by the laser unit 28 indicates a puncture depth and a blood vessel diameter in addition to the position of the puncture point. As illustrated in FIG. 5A, the laser unit 28 projects a marker consisting of two lines in a T shape on the skin surface. A point at which the two lines intersect represents a puncture point 50.

The line extending in the lateral direction from the puncture point 50 is a direction indicator 51 indicating the puncture depth and a puncture direction. The direction and length of the direction indicator 51 represent the puncture direction and the puncture depth, respectively. Here, the puncture depth is a distance from the puncture point 50 to a position of the center of gravity of the blood vessel when puncture is performed at the angle of 30°, which is the irradiation angle of the laser light, from the puncture point 50. However, as the puncture depth, a length obtained by projecting a straight line extending from the puncture point 50 to the position of the center of gravity of the blood vessel on the skin surface may be projected. For example, in a case where the puncture depth is smaller than that in the case of FIG. 5A, the direction indicator 51 is shorter as illustrated in FIG. 5B.

The line extending in the longitudinal direction from the puncture point 50 is a blood vessel diameter indicator 52 indicating the blood vessel diameter. The length of the blood vessel diameter indicator 52 represents the blood vessel diameter. For example, in a case where the blood vessel diameter is larger than that in the case of FIG. 5A, the blood vessel diameter indicator 52 is longer as illustrated in FIG. 5C.

A wavelength of the laser light emitted by the laser unit 28 only needs to be in a visible light region, and preferably can be set to green (i.e., 532 nm) which is excellent in distinguishability from blood and a skin tissue. In addition, a color may be varied depending on an indicating portion. For example, colors of the blood vessel diameter indicator 52 and the other portion can be varied. The line width of the laser light emitted by the laser unit 28 is 5 mm or less in consideration of the blood vessel diameter, preferably 1.5 mm or less, which is equivalent to an outer diameter of a puncture needle, and preferably 0.3 mm or more in consideration of ease of visual observation.

A modification of the direction indicator 51 will be described. In each of FIGS. 6A to 6C, a lower one among three direction indicators represents a deeper puncture depth. Each of the direction indicators 51 forming a marker in FIG. 6A represents the puncture depth by a length of a line as described above. Each of direction indicators 55 in FIG. 6B is a line that becomes thinner toward a distal end side (i.e., a distal end side of the arm H), and represents the puncture depth by its length. Each of direction indicators 56 in FIG. 6C represents differences in the puncture depth by different shapes having the same length.

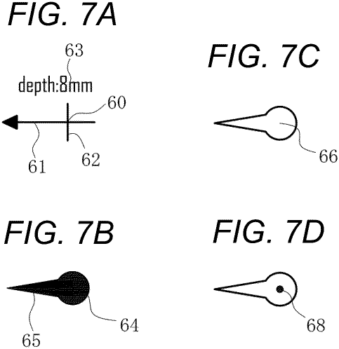

A modification of the marker projected by the laser unit 28 will be described. In FIG. 7A, a point at which a direction indicator 61 and a blood vessel diameter indicator 62 intersect represents a puncture point 60, and the direction of the direction indicator 61 represents a puncture direction. A puncture depth is represented by a depth indicator 63 arranged at the top. When the puncture depth is represented by a number (e.g., 8 mm), the operator can grasp the accurate puncture depth.

In FIG. 7B, the center of a circle represents a puncture point 64, and the diameter of the circle represents a blood vessel diameter. A direction indicator 65 has a shape that become thinner toward a distal end. As illustrated in FIG. 7C, only a contour line may be projected. In this case as well, the center of a circle is a puncture point 66. Furthermore, as illustrated in FIG. 7D, a puncture point 68 may be projected as a point. In this case, colors to be projected may vary between the point representing the puncture point 68 and the other contour line. In the other examples described hereinabove, the visibility can be improved by varying colors of the puncture point, the direction indicator, and the blood vessel diameter indicator.

The laser unit 28 can use, for example, a diffraction grating (not illustrated) in order to change the emitted laser light into any shape. The diffraction grating is formed by engraving grooves or the like on the surface of a transparent plate. The surface of the transparent plate has a region where the diffraction grating is formed and a region where the diffraction grating is not formed. When the laser light passes through the transparent plate, the emitted laser light is divided into a plurality of beams to form a shape projected on the skin surface. In this case, it is preferable to provide a plurality of the diffraction gratings or to make a position of the diffraction grating variable in order to change the shape or its size projected on the skin surface.

In order for the laser unit 28 to change a shape of the laser light, a lens (not illustrated) or a slit (not illustrated) may be used. When the lens is used, the shape of the laser light can be changed by arranging the lens between an emission position of the laser light and a projection position of the skin surface. When the lens is used, a shape to be projected is desirably a simple shape such as a cross shape or a T shape. In addition, it is preferable to provide a plurality of the lenses or make a position of the lens variable in order to change the shape to be projected. The length of the direction indicator 51 can be changed by independently moving some of the plurality of lenses.

When the slit is used, the shape of the laser light can be changed by arranging the slit between the emission position of the laser light and the projection position of the skin surface. The slit can have any shape, and is suitable for projection of a complicated shape such as an arrow shape.

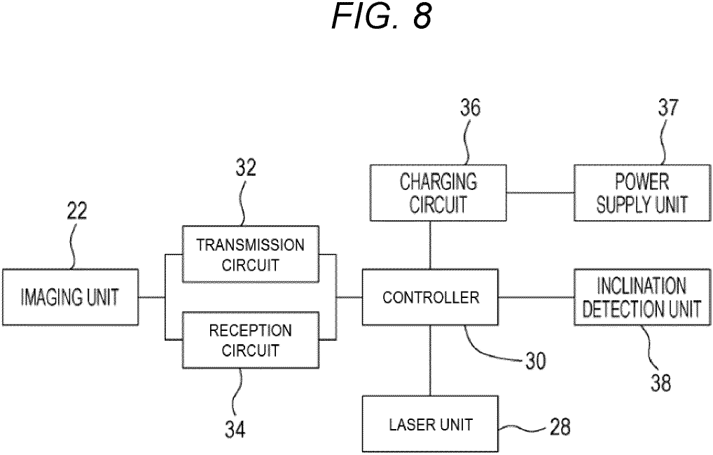

Next, a method for detecting a blood vessel position and specifying a puncture position will be described. As illustrated in FIG. 8, the blood vessel position indicating device 10 includes: the imaging unit 22 that comes into contact with a skin surface to acquire a cross-sectional image of a human body; a controller 30 that detects a blood vessel position from the cross-sectional image and calculates and determines a position of a puncture point on the skin surface from the detected blood vessel position; and the laser unit 28 that emits laser light toward the position of the puncture point detected by the controller 30 to project the puncture point on the skin surface. The controller 30 is connected to the imaging unit 22 via a transmission circuit 32 and a reception circuit 34, can cause the imaging unit 22 to acquire a cross-sectional image, and can receive the acquired cross-sectional image.

The controller 30 is connected to a power supply unit 37 including a rechargeable battery via a charging circuit 36. In addition, the controller 30 is connected to an inclination detection unit 38 including a gyro sensor.

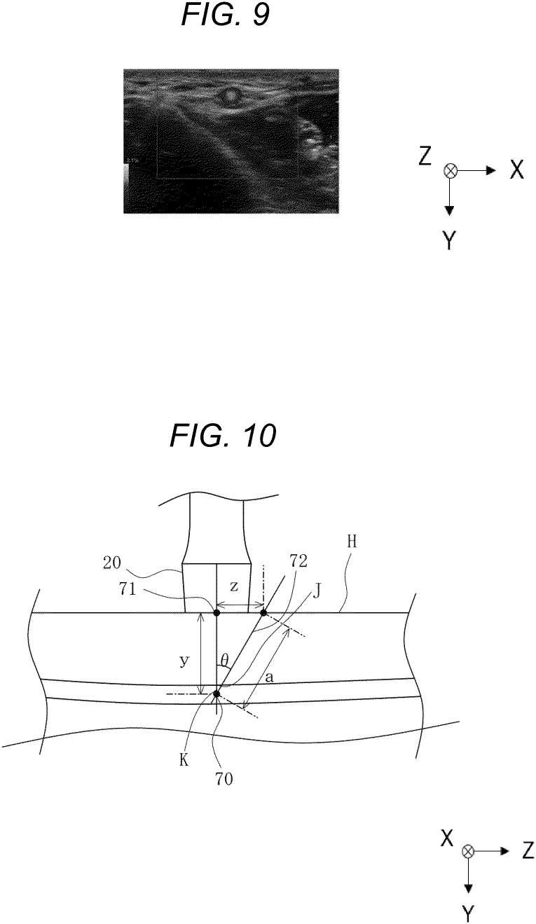

The controller 30 acquires a cross-sectional image as illustrated in FIG. 9 from the imaging unit 22. It is assumed that a lateral direction in the cross-sectional image, that is, the width direction of an arm is X direction, the longitudinal direction in the cross-sectional image, that is, the depth direction of the arm is Y direction, and the direction orthogonal to the paper surface of the cross-sectional image, that is, the length direction of the arm is Z direction. The coordinates of an upper left point in the cross-sectional image are represented by (0, 0, 0).

The controller 30 performs image analysis of the acquired cross-sectional image to detect a position of a blood vessel in the image. The controller 30 detects a region of the blood vessel in the image, and sets a position 70 of the center of gravity as the position of the blood vessel. In order to detect the region of the blood vessel in the image, it is possible to prepare a large number of images of the same type and use a machine learning or deep learning method. In addition, it is also possible to detect a region with blood flow by the Doppler method in the imaging unit 22 and recognize the region as the region of the blood vessel. When detecting the region of the blood vessel from the cross-sectional image, it is necessary to detect an artery and a vein in a distinguishable manner. The artery and the vein can be distinguished based on a position of a bone of the arm H appearing in the cross-sectional image. In addition, when the region with the blood flow is detected by the Doppler method, the artery and the vein can be distinguished by the direction of the blood flow. The coordinates of the detected position 70 of the center of gravity of the blood vessel are defined as (x, y, 0). The controller 30 also detects the diameter of the blood vessel detected from the cross-sectional image.

As illustrated in FIG. 10, the z-coordinate of the puncture position is a horizontal distance between an imaging position 71 by the imaging unit 22 and a puncture point 72, and is calculated by z = y · tanθ. In addition, the puncture depth “a” is calculated by a = y/cosθ. In this example, θ is 30°. As a result, coordinates (x, 0, z) of the puncture point 72 and the puncture depth “a” are defined. A position of the puncture point 72 corresponds to a point at which a line extending in a direction having a certain angle (30°) with respect to a perpendicular direction of the skin surface from the position 70 of the center of gravity of the blood vessel intersects with the skin surface. The controller 30 causes the laser unit 28 to emit laser light toward the calculated puncture point 72, thereby projecting a marker indicating the position of the puncture point, the puncture depth, and the blood vessel diameter. At this time, the laser unit 28 is moved to the position indicated by the x-coordinate of the puncture point 72, and then, emits the laser light. The operator can easily perform puncture by the marker projected on the skin surface.

When the blood vessel position indicating device 10 is used, the arm H can be fixed to an arm holder 40 as illustrated in FIG. 11. The arm holder 40 has a tubular base portion 41, and has an expansion/contraction portion 43 that can be expanded and contracted by air pressure in a lower portion of an inner surface 42. When the expansion/contraction portion 43 is expanded in a state in which the arm H passes through the base portion 41, the arm H is pressed against an upper wall of the inner surface 42. In this state, the upper surface of the arm H to be punctured is parallel to the inner surface 42 of the base portion 41. In this state, the imaging unit 22 can be inclined and pushed with respect to the arm H as illustrated in FIG. 11.

An inclination of the probe body 20 can be detected by the inclination detection unit 38. A reference of the inclination is the vertical direction orthogonal to the horizontal direction defined by the base portion 41. As described above, the upper surface of the arm H fixed to the base portion 41 is parallel to the inner surface 42 of the base portion 41 and is oriented along the horizontal direction, an inclination of the blood vessel position indicating device 10 with respect to the perpendicular direction of the skin surface can be detected by detecting the above inclination with respect to the vertical direction using the inclination detection unit 38. In this example, it is assumed that the inclination detection unit 38 detects that the blood vessel position indicating device 10 is inclined at an angle of φ.

In this case as well, the controller 30 first acquires a cross-sectional image from the imaging unit 22. In the cross-sectional image, Y direction is inclined at the angle of φ with respect to the perpendicular line of the skin surface. In addition, the controller 30 acquires the inclination φ of the blood vessel position indicating device 10 by the inclination detection unit 38. The controller 30 sets an upper left end position of the acquired cross-sectional image as (0, 0, 0). With the coordinates as the reference, the controller 30 detects the position 70 of the center of gravity of the blood vessel from the cross-sectional image, and sets the coordinates of the detected position 70 of the center of gravity of the blood vessel as (x, y, 0). The controller 30 also detects a diameter of the blood vessel detected from the cross-sectional image.

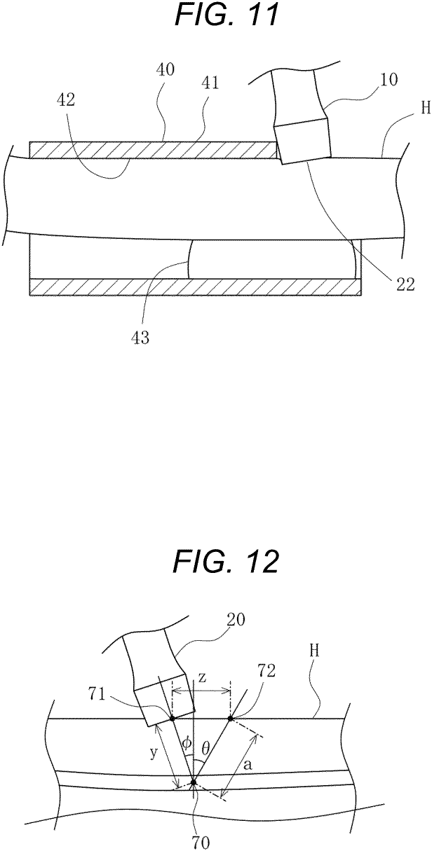

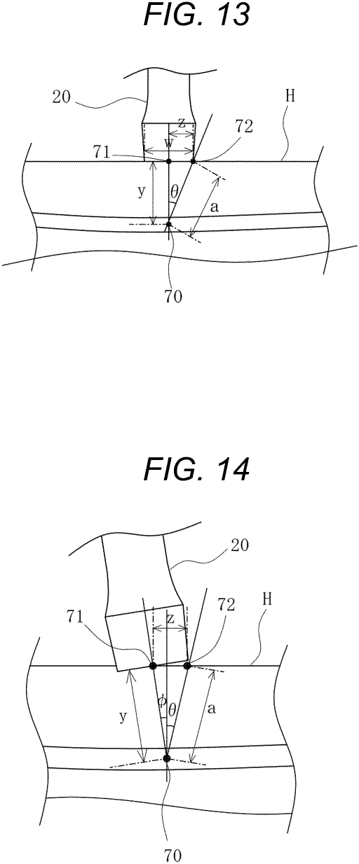

As illustrated in FIG. 12, the z-coordinate of the puncture position can be calculated by z = y (sinφ + cosφ · tanθ). In addition, the puncture depth “a” is calculated by a = y · cosφ/cosθ. As a result, coordinates (x, 0, z) of the puncture point 72 and the puncture depth a are defined. The controller 30 causes the laser unit 28 to emit laser light toward the calculated puncture point 72 and project a marker indicating the position of the puncture point, the puncture depth, and the blood vessel diameter similarly to the above example.

Although the position of the puncture point in a case where puncture is performed at the constant angle with respect to the detected position of the center of gravity of the blood vessel is projected in the example described hereinabove, the position of the puncture point may be set along one end portion of the probe body 20. In this case, a puncture angle θ differs depending on a positional relationship between the position of the center of gravity of the blood vessel and the probe body 20, and thus, it is necessary to calculate the puncture angle θ.

In this case as well, the controller 30 acquires a cross-sectional image from the imaging unit 22, and sets the upper left end position of the acquired cross-sectional image as (0, 0, 0). With the coordinates as the reference, the controller 30 detects the position 70 of the center of gravity of the blood vessel from the cross-sectional image, and sets the coordinates of the detected position 70 of the center of gravity of the blood vessel as (x, y, 0). The controller 30 also detects a diameter of the blood vessel detected from the cross-sectional image.

In a case where the lower surface of the probe body 20 is in parallel contact with the skin surface and the inclination detection unit 38 detects no inclination of the probe body 20, the z-coordinate of the puncture position is half a width W of the probe body 20 as illustrated in FIG. 13, and thus, is calculated by z = W/2. The puncture angle θ is calculated by θ = arctan(z/y). The puncture depth “a” is calculated by a = y/cosθ. As a result, coordinates (x, 0, z) of the puncture point 72 and the puncture depth a are defined. The controller 30 causes the laser unit 28 to emit laser light toward the calculated puncture point 72 and project a marker indicating the position of the puncture point, the puncture depth, and the blood vessel diameter similarly to the above example.

When the lower surface of the probe body 20 is inclined with respect to the skin surface and the inclination detection unit 38 has detected the inclination of the angle of φ with respect to the perpendicular line of the skin surface, the z-coordinate of the puncture position is calculated by z = W/2. The puncture angle θ is calculated by θ = arctan ((z - y · sinφ)/(y · cosφ)). The puncture depth a is calculated by a = y · cosφ/cosθ. As a result, coordinates (x, 0, z) of the puncture point 72 and the puncture depth a are defined. The controller 30 causes the laser unit 28 to emit laser light toward the calculated puncture point 72 and project a marker indicating the position of the puncture point, the puncture depth, and the blood vessel diameter similarly to the above example.

The direction of the blood vessel indicated as the direction indicator by the laser unit 28 can be detected by bringing the probe body 20 into contact with two or more different sites on the skin. When detecting the positions of the center of gravity of the blood vessel from cross-sectional images acquired by the imaging unit 22, the controller 30 stores the coordinates thereof. When the probe body 20 is moved to come into contact with different positions on the skin surface and the imaging unit 22 acquires cross-sectional images, the controller 30 detects the positions of the center of gravity of the blood vessel from the cross-sectional images. The controller 30 can calculate the direction of the blood vessel from differences in X direction and Y direction between the positions of the center of gravity of the blood vessel detected at the different positions. The controller 30 causes the laser unit 28 to project the calculated direction of the blood vessel as the direction indicator.

As described above, the blood vessel position indicating device 10 includes: the imaging unit 22 that comes into contact with a skin surface to acquire a cross-sectional image of a human body; the controller 30 that detects a blood vessel position from the cross-sectional image and calculates a position of a puncture point on the skin surface from the detected blood vessel position; and the laser unit 28 that emits laser light toward the position of the puncture point detected by the controller 30 to project the puncture point on the skin surface. Since the blood vessel position indicating device 10 specifies the blood vessel position with high accuracy from the cross-sectional image acquired by the imaging unit 22 and projects the puncture point on the skin surface, the operator does not need to move the line of sight to a monitor or the like, and can concentrate on the puncture operation, so that the puncture can be reliably performed regardless of the skill of the operator.

In addition, the controller 30 may detect a blood vessel diameter in addition to the blood vessel position from the cross-sectional image and calculate a puncture depth from the skin surface, and the laser unit 28 may project a marker indicating the blood vessel diameter and the puncture depth on the skin surface together with the puncture point. As a result, more information for the puncture can be transmitted to the operator, the reliability of the puncture can be further improved.

In addition, the laser unit 28 may project a direction indicator, which extends from the position of the puncture point along a direction of the puncture, on the skin surface. As a result, the direction of puncture by the operator can be reliably grasped, and the reliability of the puncture can be further improved.

In addition, the laser unit 28 may project a marker indicating the puncture depth by the length or the shape of the direction indicator. As a result, the operator can intuitively grasp the puncture depth.

In addition, the laser unit 28 may project the direction indicator to be thinner toward the distal end side. As a result, the visibility of the puncture direction can be enhanced.

In addition, the controller 30 may detect a position of the center of gravity of a blood vessel from the cross-sectional image, and may set, as the position of the puncture point on the skin surface, a point at which a line extending from the position of the center of gravity in a direction having a certain angle with respect to a perpendicular direction of the skin surface intersects with the skin surface. As a result, when a puncture angle is fixed, the position of the puncture point on the skin surface can be accurately specified.

In addition, the laser unit 28 may emit the laser light from a direction forming the puncture angle with respect to the skin surface. As a result, the operator can easily grasp the puncture angle.

In addition, the controller 30 may detect a position of the center of gravity of a blood vessel from the cross-sectional image, and may set, as the position of the puncture point on the skin surface, a point at which a line extending from the position of the center of gravity to a position of one end portion of the probe body 20 including the imaging unit 22 intersects with the skin surface. As a result, when the puncture is performed along the blood vessel position indicating device 10, the position of the puncture point on the skin surface can be accurately specified.

In addition, the controller 30 may calculate an angle formed by a line extending from the position of the center of gravity of the blood vessel to a position of one end portion of the imaging unit 22 and a perpendicular line of the skin surface as a puncture angle, and the laser unit 28 may project the puncture angle calculated by the controller 30 on the skin surface. As a result, when the puncture is performed along the blood vessel position indicating device 10, the operator can reliably grasp the puncture angle.

In addition, the controller 30 may calculate a distance from the position of the center of gravity of the blood vessel to the position of the puncture point on the skin surface as the puncture depth, and the laser unit 28 may project a marker indicating the puncture depth calculated by the controller 30 on the skin surface. As a result, the operator can grasp how much a needle needs to be inserted, and thus, more reliable puncture can be performed.

In addition, instead of calculating the position of the puncture point on the skin surface from the detected blood vessel position, the controller 30 may detect a position other than a position of the center of gravity of a blood vessel to be punctured from the cross-sectional image and calculate the position of the puncture point on the skin surface from the position other than the position of the center of gravity of the blood vessel. As a result, a distance between the puncture point and the blood vessel to be punctured is increased, and it is possible to prevent the needle from being further inserted to penetrate through the blood vessel after the blood vessel is punctured with the needle.

The invention is not limited to the above embodiments, and various modifications may be made within the technical idea of the invention by those skilled in the art. For example, the angle of the laser light emitted from the laser unit 28 coincides with the puncture angle in the above-described embodiment, but the laser light may be emitted at an angle different from the puncture angle. Since it is common recognition of medical workers that the puncture angle is about 30°, puncture can be performed even if the irradiation angle of the laser light is different from this. Since the puncture angle and the irradiation angle of the laser light are different, it is possible to prevent the laser light from being blocked by the operator’s hand, the needle used for the puncture, or the like.

In addition, a near-infrared camera and an irradiation device may be provided, and a near-infrared image may be projected onto the skin surface together with the puncture point by the laser unit 28. The near-infrared camera can capture a two-dimensional image of blood vessels of the arm, and the operator can more easily grasp an image of puncture when the two-dimensional image of the blood vessels is projected onto the skin surface.

Although a monitor that displays the cross-sectional image acquired by the imaging unit 22 is not illustrated, the blood vessel position indicating device 10 may be connected to the monitor to enable visual observation of the cross-sectional image.

In addition, the position of the center of gravity of the blood vessel to be punctured is detected from the cross-sectional image, and the position of the puncture point on the skin surface is calculated from the position of the center of gravity in the present embodiment. However, the position of the puncture point on the skin surface may be calculated by detecting a position other than the position of the center of gravity of the blood vessel to be punctured. For example, the controller 30 may detect a position K in an inner surface J of a blood vessel to be punctured located between the blood vessel and the imaging unit 22 or a membrane of the blood vessel from the cross-sectional image, and calculate the position of the puncture point based on coordinates of the position K. In addition, the controller 30 may detect the position K in the inner surface J of the blood vessel to be punctured located between the blood vessel and the imaging unit 22 or in the membrane of the blood vessel from the cross-sectional image, and calculate the position of the puncture point from coordinates of a position separated from this position by a certain distance. As a result, a distance between the puncture point and the blood vessel to be punctured is increased, and it is possible to prevent the needle from being further inserted to penetrate through the blood vessel after the blood vessel is punctured with the needle. The position separated by a certain distance is a position separated mainly in the axial direction of the blood vessel. The position may be separated in the radial direction.

At the time of puncture, a blood vessel position is sometimes changed by being pushed by the needle. In order to reliably puncture a target blood vessel, the laser unit 28 can project the direction and degree of the change in the blood vessel position.

The direction and degree of the change in the blood vessel position are detected as the controller 30 compares a stored cross-sectional image before puncture with a cross-sectional image after puncture. Thus, the controller 30 can detect the direction and degree even if the blood vessel position changes in any direction. When the direction and degree of the change in the blood vessel position are detected, the controller 30 causes the laser unit 28 to project the direction and degree of the change in the blood vessel position. The direction and degree of the change in the blood vessel position can be indicated by a line, an arrow, a numerical value, or the like. In addition, a projection mode may be changed by changing a color or shade of the projected image before and after the change in the blood vessel position.

What is claimed is:

1. A medical device for assisting in a puncture operation on a human body, comprising:
an imaging unit configured to acquire a cross-sectional image of a human body;
a laser unit configured to emit laser light and project a marker onto a skin surface of the human body; and
a controller configured to:
determine a first distance from the imaging unit to a blood vessel using the cross-sectional image acquired by the imaging unit,
determine a position of a puncture point on the skin surface with respect to the imaging unit based on the determined first distance, and
control the laser unit to project onto the skin surface a marker indicating the puncture point at the determined position thereof.

2. The medical device according to claim 1, wherein
the controller is configured to determine a diameter of the blood vessel using the cross-sectional image, and
the marker further indicates the determined diameter.

3. The medical device according to claim 2, wherein
the controller is configured to determine a puncture depth from the skin surface using the cross-sectional image, and
the marker further indicates the determined puncture depth.

4. The medical device according to claim 3, wherein the marker includes a line, a length or shape of which indicates the puncture depth.

5. The medical device according to claim 1, wherein
the controller is configured to determine a puncture direction from the puncture point to the blood vessel, and
the marker further indicates the determined puncture direction.

6. The medical device according to claim 5, wherein one end of the marker corresponding to a distal side of the human body is thinner than the other end of the marker.

7. The medical device according to claim 1, wherein
the controller is configured to determine a center of gravity of the blood vessel in the cross-sectional image, and determine a position of the blood vessel with respect to the imaging unit using the determined center of gravity,
the controller determines a distance from the imaging unit to the determined position of the blood vessel as the first distance, and
the controller determines, as the position of the puncture point, a point at which the skin surface intersects with a line extending from the position of the blood vessel toward a direction that is inclined at a predetermined angle with respect to a direction perpendicular to the skin surface.

8. The medical device according to claim 1, further comprising:
a probe body extending along a first direction and including a bottom surface along which the imaging unit is disposed, the probe body further including an extension portion that extends along a second direction crossing the first direction and includes the laser unit, wherein
the laser unit projects the marker along a direction that is inclined at a predetermined angle with respect to the first direction.

9. The medical device according to claim 1, further comprising:
a probe body including a bottom surface along which the imaging unit is disposed at a center of the bottom surface, wherein
the controller is configured to determine a center of gravity of the blood vessel in the cross-sectional image, and determine a position of the blood vessel with respect to the imaging unit using the determined center of gravity, and
the controller determines, as the position of the puncture point, a point at which the skin surface intersects a first line extending from the position of the blood vessel to one end of the bottom surface of the probe body.

10. The medical device according to claim 9, wherein
the controller is configured to determine an angle formed by the first line and a direction perpendicular to the skin surface as a puncture angle, and
the marker further indicates the puncture angle.

11. The medical device according to claim 9, wherein
the controller is configured to determine a second distance from the position of the blood vessel to the position of the puncture point as a puncture depth, and
the marker further indicates the puncture depth.

12. The medical device according to claim 1, wherein
the controller is configured to determine a position of a particular part of the blood vessel in the cross-sectional image, and determine a position of the blood vessel with respect to the imaging unit using the determined position of the particular part, and
the controller determines a distance from the imaging unit to the determined position of the particular part as the first distance.

13. A medical device for assisting in a puncture operation on a human body, comprising:
a probe body extending along a first direction and including a bottom surface, the probe body further including an extension portion that extends from the probe body toward a second direction crossing the first direction;
an imaging unit disposed along the bottom surface of the probe body and configured to acquire a cross-sectional image of a human body;
a laser unit disposed at an end of the extension portion in the second direction, and configured to project a marker onto a skin surface of the human body; and
a controller configured to determine a position of a puncture point on the skin surface using the cross-sectional image acquired by the imaging unit, and control the laser unit to project onto the skin surface a marker indicating the puncture point at the determined position thereof.

14. The medical device according to claim 13, wherein
the controller is configured to determine a diameter of the blood vessel using the cross-sectional image, and
the marker further indicates the determined diameter.

15. The medical device according to claim 14, wherein
the controller is configured to determine a puncture depth from the skin surface using the cross-sectional image, and
the marker further indicates the determined puncture depth.

16. A method for assisting in a puncture operation on a human body using a medical device that includes:
an imaging unit configured to acquire a cross-sectional image of a human body, and
a laser unit configured to emit laser light and project a marker onto a skin surface of the human body, the method comprising:
determining a first distance from the imaging unit to a blood vessel using the cross-sectional image acquired by the imaging unit;
determining a position of a puncture point on the skin surface with respect to the imaging unit based on the determined first distance; and
controlling the laser unit to project onto the skin surface a marker indicating the puncture point at the determined position thereof.

17. The method according to claim 16, further comprising:
determining a diameter of the blood vessel using the cross-sectional image, wherein
the marker further indicates the determined diameter.

18. The method according to claim 17, further comprising:
determining a puncture depth from the skin surface using the cross-sectional image, wherein
the marker further indicates the determined puncture depth.

19. The method according to claim 18, wherein the marker includes a line, a length or shape of which indicates the puncture depth.

20. The method according to claim 16, further comprising:
determining a puncture direction from the puncture point to the blood vessel, wherein
the marker further indicates the determined puncture direction.
